(19) United States
(12) Patent Application Publication
(10) Pub. No.: US 2026/0278558 A1
(43) Pub. Date: Sep. 17, 2026

(54) A SYSTEM AND METHOD FOR MANAGING HEALTHCARE SERVICES AND NETWORKS

(71) Applicant: SEEYOUDOC CORPORATION, Marikina (PH)

(72) Inventor: Noel Gary DEL CASTILLO, Marikina (PH)

(21) Appl. No.: 18/868,511

(22) PCT Filed: Apr. 26, 2024
(86) PCT No.: PCT/PH2024/050007

§ 371 Date: Nov. 22, 2024

(30) Foreign Application Priority Data
Apr. 27, 2023 (PH) 12023050167

(51) Int. Cl. G06Q 10/1093; G16H 40/20

(52) U.S. Cl. CPC G06Q 10/1093; G16H 40/20

(57) ABSTRACT

The present invention relates to a patient-centric integrated system for managing a plurality of healthcare services and networks. The system is configured to receive an appointment request from the patients user group, transmit the appointment request to the associated doctor, establish a consultation, whether virtual or onsite, between the associated doctor and the patient, provide a means for the sending and receiving of medical-related documents between the associated doctor and the patient, and provide a means for a clinic, a hospital, and/or a healthcare provider network (HCPN) linked to the associated doctor to manage the appointments, the virtual waiting rooms, the collection of fees, the referral systems, and the sending of service orders within the network.

### TECHNICAL FIELD OF THE INVENTION

The present invention relates generally to healthcare information and communication technologies and more particularly, to systems and computer-implemented methods for managing healthcare services and networks.

### BACKGROUND OF THE INVENTION

Telemedicine has become an increasingly known alternative to the traditional onsite consultations. Telemedicine platforms generally provide a means for the patients to conveniently request for and establish a real-time remote consultation with a healthcare professional, typically, via a video conference.

One such platform is disclosed in U.S. Pat. No. 11,482,326 B2 ('326) which aims to provide a web-based application for conducting remote counselling sessions by means of various mobile devices. The application according to '326 allows the client to access and log into a website, search a database for online experts, and select an appropriate expert for the counselling session. The selected healthcare provider may also refer the patient to another healthcare provider inside the platform. A two-way audiovisual communication session is then initiated between the patient computing device and the provider computing device. This referral process can automatically transfer the user's files, documents, history and/or data to the referred expert and may allow the referred expert to request for user authorization to access said files.

Another relevant technology is presented in US 2018/0096483 A1 ('483) which likewise provides the patients a means to schedule appointments with a health care practitioner (HCP) via their computer. Particularly, the technology according to '483 is directed to enhancing the method of information presentation during referral-based appointments. This is accomplished through the steps of creating patient health data for a patient, storing the patient health data on a server, accessing the patient health data by one of the patient and a health care provider on a corresponding computer connected over the internet, and presenting the patient health data accessed in sections searchable by icons and with transcribable portions and which may be presented in graphical forms.

The technologies according to '326 and '483, however, are limited to the clinical operations, such as the diagnosis, issuance of prescriptions, and interactions between the patient and the healthcare professional and does not take into account the more complex management issues in terms of the healthcare professional's type of medical practice, particularly, the affiliation to networks such as a clinic, a hospital or a healthcare provider network (HCPN).

A healthcare practitioner may be in private practice or associated with other healthcare practitioners in a clinic, in a hospital-based employment, or in a combination of any of these. For instance, a healthcare practitioner may be affiliated to multiple clinics, to a hospital, and to an HCPN. The cited technologies, as well as the existing systems in the market, do not offer integrated healthcare services with telemedicine within the hospital level. There is therefore a need to consider this wide range of medical practices in order to properly manage the availability of practitioners and services that the patients can choose so as to avoid booking issues (e.g, double-booking), as well as to provide a means for the professional and other medical fees to be properly distributed among the necessary networks, which depends on the healthcare practitioner's affiliation to the various levels of organizations.

Additionally, the healthcare practitioner and the patient may have preferences in terms of the mode of consultation-which may be conducted onsite or online. Hence, to manage the healthcare services and networks more efficiently, there is also a need to incorporate a means to monitor transactions regardless of the mode of consultation chosen.

On the other hand, HCPNs may be classified as public, private, or mixed healthcare providers. Public HCPNs may be further classified as province-wide which may include the provincial, municipal, and city health offices (CHOs), hospitals, health centers, health stations, and other health facilities and services under the local government units (LGUs). It can also be considered as city-wide which may include CHOs, hospitals, health centers, barangay health units (BHUs), and other city-level health facilities and services.

HCPN-level of information provides data that can be used for epidemiology, biostatistics, and other related disciplines that promote public health. Population health monitoring plays a crucial role in public health as it allows the identification and diagnosis of community health problems and hazards, particularly in terms of quantification of the extent of diseases and in the identification of patterns of change and associated risk factors (Tulchinsky and Varavikova, 2014).

Data for population health monitoring can be extracted from tools that properly incorporate HCPN-levels of information without compromising the privacy of its users.

One technology, as disclosed in U.S. Pat. No. 10,839,964 B2 ('964) provides a means to monitor healthcare information via a cloud-based clinical information system wherein credentials and/or rules for accessing and sharing (e.g., viewing, receiving, downloading, uploading, sending, etc.) the information is governed according to a hierarchical organizational scheme. The entity types in the hierarchical organizational scheme according to '964 may include communities (e. g, a plurality of research collaborators), integrated delivery networks (IDN) (e.g, a plurality of facilities and/or providers having business and/or legal relationships), sites (e.g, hospitals, imaging centers, etc.), groups (e.g, a plurality of clinicians), clinicians, and patients.

The technology according to '964, however, is not particularly concerned with methods relating to the conducting and managing of virtual consultations and managing of virtual facilities across the different levels of network. For instance, HCPNs, as in the case of LGUs, would need a system that allows them to manage integrated telemedicine services within the city wide or barangay levels and to issues referrals while CHOs would need a system to monitor the performances of their healthcare workers.

Additionally, there is also a need for insurance corporations to facilitate the funds being transferred to their accredited HCPNs and for the conducting of medical consultations to be seamlessly integrated into a system that takes into account the various medical practices of the healthcare providers.

There is therefore a need to build a patient-centric integrated system to help healthcare provider networks to offer telemedicine services online and onsite, track service performance, provide regulatory reports, manage a seamless integration of healthcare services within a network of doctors, clinics, and medical facilities, and facilitate the funds being transferred from insurance corporations to their accredited HCPNs, and monitor the associated impacts.

### SUMMARY OF THE INVENTION

Accordingly, the present invention aims to build the overall supply chain of Universal Healthcare from healthcare provider networks (HCPN), medical facilities, clinic, doctors, and patients while complying with the requirements of Health Level Seven (HL7®) Fast Healthcare Interoperability Resource (FHIR®) standards and Health Insurance Portability and Accountability Act (HIPAA), by providing an integrated online platform that allows HCPN to manage their network, publish healthcare services, provide seamless healthcare services within its network via a referral system, and monitor performances of all their service providers within the network.

In one aspect of the present invention, there is provided a system for managing a plurality of healthcare services and networks comprising a plurality of devices configured to receive input from a plurality of user groups and an application cluster deployed in a private network configured to run a plurality of computer-implemented instructions to interact with the plurality of user groups via a plurality of user interfaces, wherein the plurality of user groups comprises a plurality of patients, doctors, clinic administrators, hospital managers and HCPN administrators in varying degrees of association with one another.

In another aspect of the present invention, there is provided a computer-implemented method for managing a plurality of healthcare services and networks by retrieving and processing an appointment request from a patient, transmitting said appointment request to a doctor, retrieving and processing a response from the doctor, establishing a virtual consultation between the patient and the doctor, and retrieving further management actions from and distributing fees between the doctor and any of the associated user groups, such as the clinic administrators, the hospitals managers, and the HCPN administrators, to which the doctor is affiliated, according to a predetermined set of rules.

In yet another aspect of the present invention, there is provided a computer-implemented method that provides a means for the different network levels to manage both online and onsite consultations and both advanced and walk-in appointments.

### BRIEF DESCRIPTION OF THE DRAWINGS

FIG. 1 shows the system architecture of the present invention.

FIG. 2 shows an embodiment of the interaction between the system and the user groups of the present invention.

FIG. 3 shows an embodiment of the possible user associations between the preferred user groups.

FIG. 4 shows another embodiment of the interaction between the system and the user groups of the present invention.

FIG. 5 shows the interaction between the patient user group and the medical practitioners user group using the present invention.

FIG. 6 shows the user flow on the day of appointment.

### DETAILED DESCRIPTION OF THE INVENTION

The present invention refers to a system and a method for managing a plurality of healthcare services and networks, which may be implemented in the form of a web-based application or a mobile application.

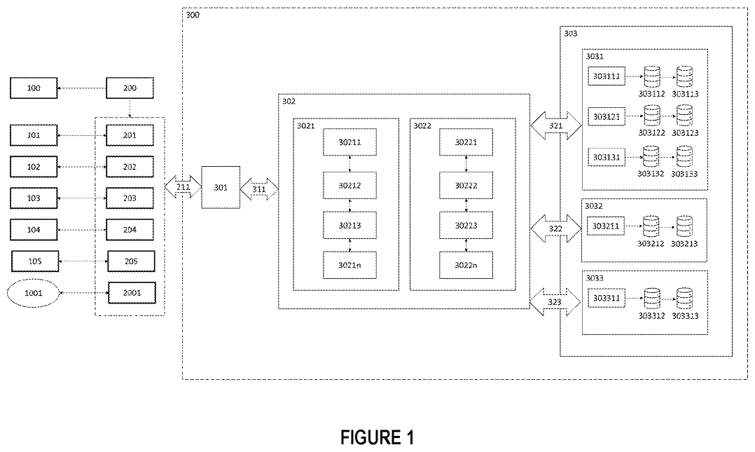

As shown in FIG. 1, an embodiment of the present invention deployed in a private network 300 may be accessed by a plurality of user groups 100 using a device comprising a module for connecting to an external network via the internet. The external network is publicly accessible and uses the domain name system (DNS) 200 for identifying the IP address requested by the plurality of user groups 100.

The DNS 200 further comprises a plurality of subdomains. In a preferred embodiment, the first user group 101 may request access to the private cloud using the first subdomain 201, the second user group 102 using the second subdomain 202, the third user group 103 using the third subdomain 203, the fourth user group 104 using the fourth subdomain 204, and the fifth user group 105 using the fifth subdomain 205.

Additionally, a sixth user group 1001 may comprise a plurality of third party applications, or a plurality of users relating to third party applications, configured to connect to an API 2001 of the present invention for access request. A communication protocol 211 is then configured to send the access request to the private network 300. The communication protocol 211 may be in the form of Hypertext Transfer Protocol Secure (HTTPS) or WebSocket Secure (WSS).

An application programming interface (API) object 301, which is a reverse proxy web server, communicates via the communication protocol 211 to receive the access request from the plurality of user groups 100. The API object 301 then sends said requests to a destined node in the private network 300 according to a predetermined set of rules.

Deployed in the private network 300, the system according to the present invention comprises an application cluster 302 configured to utilize a plurality of shared libraries 303 as needed based on its set of instructions. The application cluster 302 comprises a set of instructions stored in a non-transitory computer-readable medium and executable in at least one processor. Particularly, the application cluster 302 comprises a first service 3021 configured to run a first set of docker containers 30211, 30212, 30213, . . . ,3021n for managing online processes and a second service 3022 configured to run a second set of docker containers 30221, 30222, 30223, . . . ,3022n for managing background processes.

The application cluster 302 communicates with the plurality of shared libraries 303 via a plurality of communication protocols. Particularly, the application cluster communicates with the first service 3031 via a first communication protocol, with the second service 3032 via a second communication protocol 322, and with the third service 3033 via a third communication protocol 323. Preferably, all of the communication protocols 321, 322, and 323 are in the form of Transmission Control Protocol (TCP).

The plurality of shared libraries 303 are accessible to the application cluster 302. The first service 3031 is a database pool comprising a first shared library 303111, a second shared library 303121, and a third shared library 303131.

The first shared library 303111 is dedicated for handling a plurality of authentication and authorization functionalities and which is configured to record a plurality of information using the first master database 303112, which in turn is configured to connect to the first backup database 303113.

The second shared library 303121 is dedicated for handling a plurality of core business functionalities and which is configured to record a plurality of information using the second master database 303122, which in turn is configured to connect to the second backup database 303123.

The third shared library 303131 is dedicated for handling a plurality of additional functionalities and which is configured to record a plurality of information using the third master database 303132, which in turn is configured to connect to the third backup database 303133. In the preferred embodiment, the plurality of additional functionalities are functionalities added in order to adhere to the requirements of Health Level Seven (HL7®) Fast Healthcare Interoperability Resource (FHIR®) standards.

The second service 3032 comprises at least one shared library, comprising a search module 303211 dedicated for handling the search functionalities and which is configured to record a plurality of information using the search master database 303212, which in turn is configured to connect to the search backup database 303213.

The third service 3033 comprises at least one shared library, comprising a queuing module 303311 dedicated for handling a virtual waiting room and which is configured to record a plurality of information using the queuing master database 303312, which in turn is configured to connect to the queuing backup database 303313.

In a preferred embodiment, the application cluster 302 is further configured to communicate with a fourth service for storing static files and with a fifth service for generating and recording real-time communications such as a video call (not shown).

Users from the plurality of user groups 100 may create a personal account and access their accounts using a plurality of devices configured to receive input from said plurality of user groups and to connect to an external network via the internet.

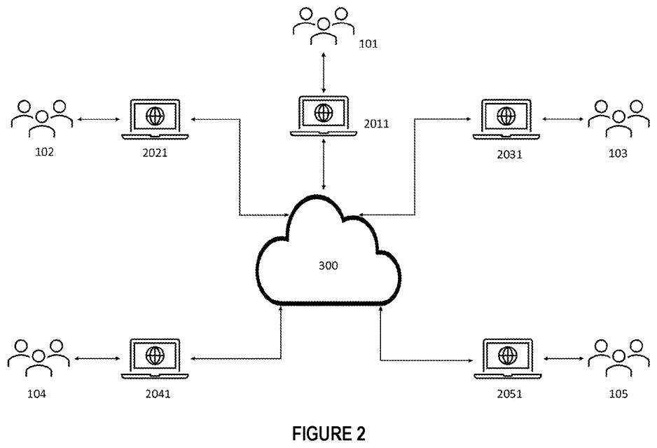

In the preferred embodiment shown in FIG. 2, there are five user groups, comprising at least one user, who can access the system deployed in the private network 300. The at least one user in the first user group 101 interacts with the system and with other users via the first user interface 2011, the at least one user in the second user group 102 via the second user interface 2021, the at least one user in the third user group 103 via the third user interface 2031, the at least one user in the fourth user group 104 via the fourth user interface 2041, and the at least one user in the fifth user group 105 via the fifth user interface 2051.

In this preferred embodiment, the first user group 101 comprises a plurality of patients, the second user group 102 comprises a plurality of healthcare practitioners or medical doctors, the third user group 103 comprises a plurality of clinic administrators, the fourth user group 104 comprises a plurality of hospital managers, and the fifth user group 105 comprises a plurality of HCPN administrators.

The terms “healthcare practitioners” and “doctors” are used interchangeably in this document. Meanwhile, the term “clinic administrators” may refer to users who assist the doctors in planning the clinic or to users who manage a triage clinic. On the other hand, the term “hospital managers” may refer to representatives from medical facilities such as hospitals. Finally, the term “HCPN administrators” may refer to representatives from agencies under the Department of Health, LGUs, private medical groups, health maintenance organizations (HMOs), and the likes.

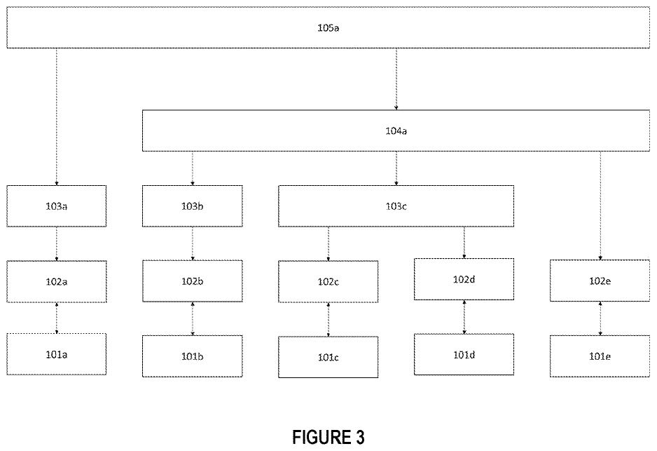

A user from a certain user group may be associated with another user or users in another user group. As shown in an embodiment in FIG. 3, patients 101a, 101b, 101c, 101d, and 101e may directly or indirectly (i.e, by selecting an associated healthcare service) select any of the healthcare practitioners 102a, 102b, 102c, 102d, and 102e of the second user group 102.

If the at least one user in the second user group 102 is affiliated with at least one clinic, said user may be associated with at least one user in the third user group 103, as in the healthcare practitioner 102a being associated with clinic administrator 103a, healthcare practitioner 102b being associated with clinic administrator 103b, and healthcare practitioners 102c and 102d being associated with clinic administrator 103c.

Alternatively, any of the healthcare practitioners in the second user group 102 may be associated with more than one users in the third user group 103.

The present invention allows the healthcare practitioners in the second user group 102 and the clinic administrators in the third user group 103 to offer and conduct healthcare services online and onsite.

The application cluster 302 is also configured to provide users in the third user group 103 (i.e, the clinic administrators) a means to add or remove associated users from the second user group (i.e, the associated doctors) and to manage a virtual clinic. There are two types of virtual clinics. The first is the general clinic wherein the clinic administrators 103 may assist the doctors 102 in manning the clinic while the second is the triage clinic wherein the clinic administrators 102 may collect preliminary information and assess the patient's needs through chats.

If the at least one user in the third user group 103 is affiliated with a hospital, said user may be associated with at least one user in the fourth user group 104, as in clinic administrator 103b and 103c being associated with hospital manager 104a.

The application cluster 302 is further configured to provide users in the fourth user group 104 (i.e, hospital managers) a means to add or remove associated users from the third user group (i.e, clinic administrators) 103 and associated users from the second user group (i.e, doctors) 102.

Private hospitals may benefit from the present invention by assigning one user in the fourth user group 104 who may act as the hospital manager. Said hospital manager may, via the fourth user interface, set virtual clinics per department and assign affiliated doctors who will be providing healthcare services, both online and onsite. Moreover, the hospital manager may further set up an account for each of the clinics or departments by assigning a user in the third user group 103 who may act as the clinic administrator via the third user interface 2031.

If the at least one user in the third user group 103 is affiliated with an HCPN, said user may be associated with at least one user in the fifth user group 105, as in hospital manager 103a being associated with HCPN administrator 105a.

If the at least one user in the fourth user group 104 is affiliated with an HCPN, said user may be associated with at least one user in the fifth user group 105, as in hospital managers 104a and 104b being associated with an HCPN administrator 105a.

For instance, LGUs may assign a user in the fifth user group 105 to act as an HCPN administrator and the administrators in its BHUs as affiliated users in the third user group 103 to act as clinic administrators.

In this embodiment, the application cluster 302 is further configured to provide users in the fifth user group 105 (i.e, HCPN administrators) a means to manage a plurality of users and a plurality of information from the fourth user group (i.e, hospital managers) 104 and a plurality of users from the third user group (i.e, clinic administrators) 103 in the form of service orders.

The application cluster 302 is further configured to display an analytics dashboard in the second user interface 2021 using any information associated to the at least one user in the second user group 102, in the third user interface 2031 using any information associated to the at least one user in the third user group 103, in the fourth user interface 2041 using any information associated to the at least one user in the fourth user group 104, and in the fifth user interface 2051 using any information associated to the at least one user in the fifth user group 105.

The application cluster 302 is configured to monitor the activities of the plurality of user groups 100, extract data from said activities according to a predetermined set of instructions, and present said data in the form of visual elements like charts, graphs, maps, and other data visualization tools. The analytics dashboard may be helpful to the users in terms of monitoring appointments, patient demographics, revenues, and patient feedback.

For instance, users in the fifth user group 105, such as the administrators from the LGUs, may monitor the performances of the doctors in the CHOs and BHUs using the analytics dashboard.

The analytics dashboard may be located in another subdomain and may function as an add-on subscription to the second user group 102 (i.e, the doctors), third user group 103 (i.e, clinic administrators), fourth user group 104 (hospital managers) and fifth user group 105 (i.e, HCPN administrators).

Other possible user associations that are not beyond the scope of the present invention may be included in the actual implementation of the present invention.

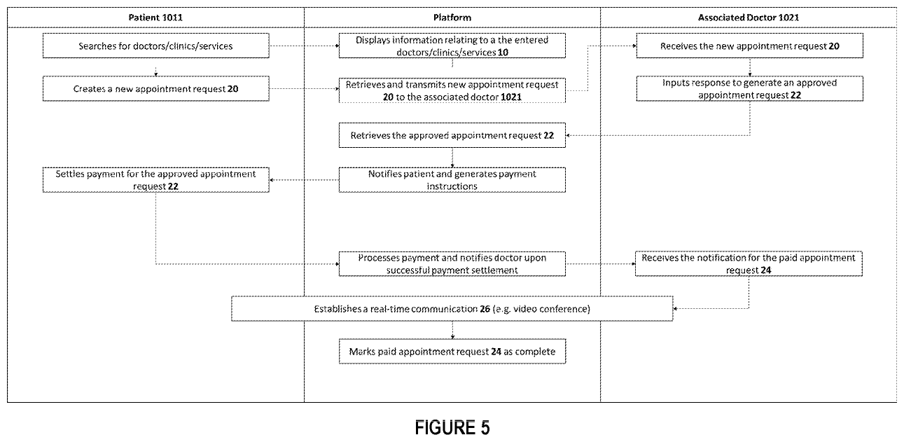

In another preferred embodiment, as shown in FIG. 4, the plurality of user groups 100 further comprises a sixth user group 106 communicating via a sixth user interface 2061, a seventh user group 107 communicating via a seventh user interface 2071, and an eighth user group 108 communicating via an eighth user interface 2081. In this preferred embodiment, the sixth user group 106 comprises a plurality of medical societies administrators, the seventh user group 107 comprises a plurality of medical partners, and the eighth user group 108 comprises a plurality of administrators for virtual facilities.

Additionally, if the at least one user in the second user group 102 is affiliated with a medical society, said user may be associated with at least one user in the sixth user group 106. For instance, the application cluster 302 provides the at least one user in the sixth user group (i.e, the medical society user) with a means to track a doctor's membership, collect membership fees, create events and issue Continuing Professional Development (CPD) credits via a unique Quick Response (QR) code.

If the at least one user in the fourth user group 104 has a medical partner who is part of the seventh user group 107, then said user in the fourth user group 104 may be associated with the said user in the seventh user group 107. For instance, the application cluster 302 provides the at least one user in the seventh user group 107 (e.g, a medical partner) to earn commissions once a user in the fourth user group 104 (e.g, hospital manager) is referred.

The at least one user from the eighth user group 108 may access a medical analytics for virtual facilities using the eighth user interface 2081. The application cluster 302 may be further configured to monitor the activities of the plurality of user groups 100, extract data from said activities according to a predetermined set of instructions, and present said data in order to provide the at least one user from the eighth user group 108 with real-time reports, patient and doctor demographics, appointment heat maps, revenue reports, information on the ratings and complaints, and the likes.

All user groups follow an onboarding process before being able to use the present invention. Specifically, doctors and healthcare providers should initially register their businesses and officered services, preferably, using the first subdomain 201.

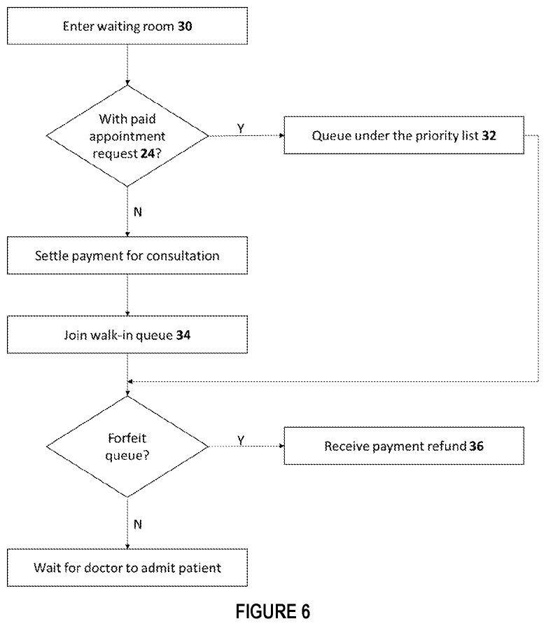

FIG. 5 shows the preferred embodiment of the process flow for an approved appointment request and the interaction between the at least one user from the patient user group and the at least one user form the medical practitioner or doctor user group 1021. Upon creating an account, the patient user 1011 may setup, view, and update multiple subprofiles registered under the account (e.g. profiles for family members). The patient user 1011 may also upload or delete medical records associated with any of the multiple profiles.

The patient user 1011 may also view and cancel or reschedule any of the appointments associated with the user account. In order to create an appointment, the patient may browse through a displayed list of available doctors, clinics, and/or services, or preferably, may search for the preferred doctor, clinic, or service. Then the application cluster, using the search module 303211, retrieves the query and displays the information relating to the query. The patient user 1011 then creates a new appointment request 20, which is then retrieved and transmitted to the associated doctor 1021 for approval. The associated doctor 1021 receives the appointment request and inputs response.

The associated doctor 1021 may reschedule, cancel, or approve the appointment. In any of these cases, the patient user 1011 receives a notification. In the event that the associated doctor 1021 reschedules the appointment by setting a proposed new schedule, the application cluster 302 provides the patient user 1011 a means to accept or decline the proposed new schedule. In another embodiment, the doctor may refer the appointment to other doctor users.

Upon the associated doctor's approval, the application cluster 302 retrieves the response and notifies the patient of an approved appointment request 22 and generates a set of payment instructions. The patient user 1011 then settles payment for the approved appointment request. If one embodiment, if another patient user was approved and paid for the same appointment request, the patient user 1011 will be notified of an unsuccessful appointment request after which a new schedule may be requested by the patient user 1011. Upon successful payment, the application cluster 302 notifies the associated doctor 1021 regarding the paid appointment request 24. The application cluster 302 processes the plurality of appointment information based on the paid appointment request 24 and establishes a real-time communication 26 (e.g. video conference) between the patient user 1011 and the associated doctor 1021. Upon completion, the application cluster 302 marks the appointment as complete.

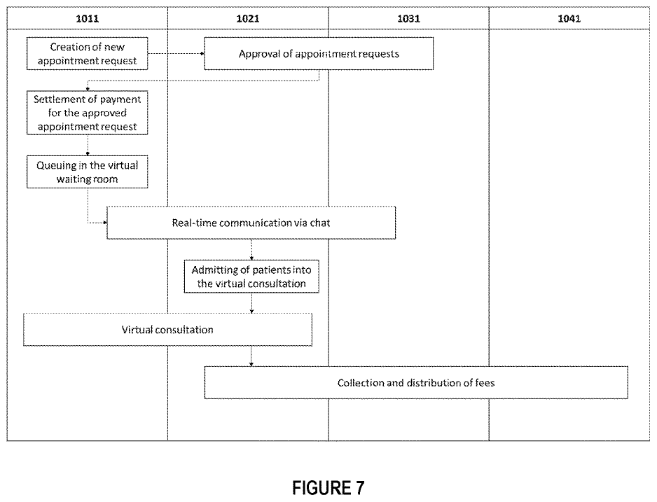

Alternatively, a patient may also choose a walk-in remote consultation session. FIG. 6 shows an embodiment of the user flow on the appointment date. The patient user 1001 enters the waiting room 30. If with a paid appointment request 24, the patient user 1001 is queued under the priority list 32. However, if no prior appointment request was paid, the patient user 1001 may settle the consultation fees and join the walk-in queue 34. The patient user 1001 may choose to forfeit the queue and receive payment refund 36.

The application cluster 302 may be further configured to provide a means to play videos or display any form of multimedia on the user's screen while inside the waiting room 30.

The virtual consultation commences once the associated doctor 1021 admits the next patient in queue. In the preferred embodiment, the application cluster is further configured to provide a real-time communication platform, such as via chat, between the associated doctor 1021 and the patients and/or other visitors in the waiting room 30.

The real-time communication 26 is, preferably, in the form of a video conference, or alternatively, in the form of a chat.

During the consultation, the patient user 1011 may add additional medical files, record the session for personal use, avail add-on services, or prematurely end the appointment at any time.

On the other hand, the associated doctor 1021 may also record the session, create a medical case for the appointment, use medical templates for medical documents, view medical files of the admitted patient user 1011, refer the patient user 1011 for another appointment consultation, and end the virtual consultation.

The patient user 1011 and the associated doctor 1021 may perform other actions relating to the functionalities of video conferencing and chatting platforms not mentioned above but are well-known in the art, such as turning on and off of audios and videos, and the likes.

In a preferred embodiment, upon the completion of the consultation, the application cluster 302 may be configured to provide a platform for retrieving customer feedback from the patient user 1011, paying of any availed add-on services, receiving relevant notifications relating to actions initiated by the associated doctor 1021, such as the transmittal of medical documents and referrals to another doctor or specialty.

In another preferred embodiment, the application cluster 302 may further be configured to provide a platform for retrieving feedback from the associated doctor 1021, sending of official medical records to the patient user 1011 such as an electronic prescription, laboratory test requests, and the likes, reviewing and making additional modifications to the patient user's 1101 medical notes or medical case.

In another preferred embodiment, the application cluster 302 is further configured to provide the associated doctor 1021 a means to manually mark an appointment as complete once an onsite consultation took place instead of the virtual consultation.

In the preferred embodiment, the application cluster 302 is further configured to generate an email summary of the consultation performance, such as the completed appointments, referrals, and earnings, of the associated doctor 1021 and display it via the second user interface 2021 for the associated doctor to view. In yet another preferred embodiment, the application cluster 302 is further configured to collect fees from the patient user 1011 and transmit it to the associated doctor 1021. In another embodiment, the application cluster 302 is further configured to allow the associated doctor 1021 to waive the fees or to apply discounts according to a predetermined set of rules.

In the event that the associated doctor is affiliated with an associated clinic administrator 1031, the application cluster is further configured to distribute fees between the associated doctor 1021 and the associated clinic administrator 1031 according to a predetermined set of rules.

The application cluster 302 is further configured to retrieve a plurality of user information and a plurality of information relating to the actions made by the associated clinic administrator 1031 via the third user interface 2031. In the preferred embodiment, these actions may refer to a plurality of service orders such as adding or deleting doctors from the list of doctors associated with the clinic administrator account, viewing, reviewing, and approving of appointments on behalf of the associated doctor 1021, receiving appointment referrals and assigning them to any of the doctors affiliated to the clinic, collecting fees as distributed according to a predetermined set of rules, viewing the total earnings, adjusting clinic settings, and tracking the performance via a dashboard module. Moreover, the clinic administrators of the third user group 103 act as a middle ground between the doctor and the hospital and can take service orders from any associated hospital manager 1041.

In the preferred embodiment, as shown in FIG. 7, the application cluster is further configured to provide a platform for establishing a real time communication, such as via chat, between the clinic administrator 1031 and the patients in the waiting room 30.

In the preferred embodiment, in the event that the associated doctor is further affiliated with an associated hospital manager 1041, the application cluster 302 is further configured to distribute fees among the associated doctor 1021, the associated clinic administrator 1031, and the associated hospital manager 1041, according to a predetermined set of rules.

Upon being onboarded, the at least one user in the fourth user group 104 may start to access a user account via the fourth subdomain 204. The account of the at least one user in the fourth user group 104 may contain a plurality of subprofiles for a facility users. The application cluster 302 is configured to retrieve a plurality of user information and a plurality of information relating to the actions made by the at least one user in the fourth user group 104 via the fourth user interface 2041. These actions may relate to the management of the plurality of facility users, the plurality of associated clinics, and the plurality of service requests for referrals. Particularly, the at least one user in the fourth user group may create service orders to be issued to its associated users in the third user group 103.

The fourth user interface 2041 may further comprise a front desk systems wherein the patients in the first user group 101 may reach out to a frontdesk receptionist, which may be one of the subprofiles within the account of the at least one user in the fourth user group 104. The frontdesk receptionist may refer a patient to at least one user in the second user group 102 or third user group 103 using an intuitive referral system.

Finally, the at least one user in the fifth user group 105 may start to access a user account via the fifth subdomain 205. The application cluster 302 is configured to retrieve a plurality of user information and a plurality of information relating to the actions made by the at least one user in the fifth user group 105 via the fourth user interface 2051. These actions may comprise a plurality of service orders which may be in the form of announcements (e.g. important announcements and written memorandums) or time-sensitive action items (e.g. doctor referrals, removing or adding of affiliated users).

During the transaction of the at least one user in the first user group 101, the application cluster 302 is configured to identify which the users from the second user group 102, third user group 103, fourth user group 104 and fifth user group 105 are associated with the transaction of the at least one user in the first user group. Then the application cluster 302 is configured to determine which of the plurality of user information and plurality of information relating to the actions made by the users in the first user group 101 is transmitted or is accessible to the associated users in the second user group 102 (i,e, first set of information), which is transmitted or accessible to the associated users in the third user group 103 (i.e, second set of information), which is transmitted or accessible to the associated users in the fourth user group 104 (i.e, third set of information), and which is transmitted or accessible to the associated users in the fifth user group 105 (i.e, fourth set of information). This is done to comply with the standards for exchange, integration, sharing and retrieval of electronic health information according to the Health Level Seven (HL7®) Fast Healthcare Interoperability Resource (FHIR®) and Health Insurance Portability and Accountability Act (HIPAA).

The present invention incorporates a hierarchical level of access from the second user group 102 to the fifth user group 105 by use of service orders. The application cluster 302 is configured to receive a service order from a user group with a higher level, transmits it to the intended user or users within the network, and provides a means for the receiver to accomplish the service order.

For instance, users within the fifth user group 105 may issue service orders to a first set of afflated users comprising any of the users in the fourth user group 104, third user group 103, or second user group 102. Accordingly, users within the fourth user group 104, third user group 103, and second user group 102 may receive and respond to the service orders from the associated user in the fifth user group 105. In turn, users within the fourth user group 104 may issue service orders to a second set of affiliated users comprising any of the users in the third user group 103 and in the second user group 102. In turn, the users in the third user group 103 and second user group 102 may receive and respond to the service orders from the associated user in the fourth user group. Ultimately, users in the third user group may issue service orders to a third set of affiliated users comprising any of the users in the second user group 102.

Using the present invention as described, the management aspects of healthcare can be seamlessly integrated with the clinical operations. Ultimately, the present invention allows healthcare networks to maintain a health information system which incorporates electronic health records, electronic prescription logs, and the likes, while ensuring patient privacy and confidentiality, in compliance with the Data Privacy Act and the Universal Health Care Act.

The preferred embodiment of this invention is described in the above-mentioned detailed description. It is understood that those skilled in the art may conceive modifications and/or variations to the embodiment shown and described therein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art. The foregoing description of a preferred embodiment and best mode of the invention known to the applicant at the time of filing the application has been presented and is intended for the purposes of illustration and description. It is not intended to be exhaustive or to limit the present invention to the precise form disclosed, and many modifications and variations are possible in the light of the above teachings.

1. A system for managing a plurality of healthcare services and networks comprising:
a plurality of devices configured to receive input from a plurality of user groups, said plurality of devices comprising a module for connecting to an external network via internet;
an application cluster deployed in a private network, said application cluster comprising a plurality of instructions stored in a non-transitory computer-readable medium and executable in at least one processor;
an application programming interface (API) object configured to receive input from the external network and send said requests to a destined node in the private network;
a first user interface for receiving an appointment request from at least one user in a first user group;
a second user interface for receiving a response to the appointment request from at least one user in a second user group associated with the appointment request;
a means for facilitating a real-time communication between the at least one user in the first user group and the at least one associated user in the second user group according to a predetermined set of rules;
characterized in that the application cluster configured to:
communicate with at least one user in a third user group via a third user interface;
communicate with at least one user in a fourth user group via a fourth user interface;
communicate with at least one user in a fifth user group via a fifth user interface,
characterized in that the application cluster is further configured to:
provide a means for the at least one user in the fifth user group to issue a plurality of service orders to a first set of affiliated users comprising a plurality of users from the fourth user group, a plurality of users from the third user group, and/or a plurality of users from the second user group;
provide a means for the at least one user in the fourth user group to issue a plurality of service orders to a second set of affiliated users comprising a plurality of users from the third user group and/or a plurality of users from the second user group;
provide a means for the at least one user in the third user group to issue a plurality of service orders to a third set of affiliated users comprising a plurality of users from the second user group; and
provide a means for the first set of affiliated users, the second set of affiliated users, and the third set of affiliated users to respond to the service orders received.

2. The system according to claim 1, characterized in that the application cluster is further configured to collect fees from the at least one user in the first user group upon completion of the real-time communication and distribute the collected fees to the at least one associated user in the second user group.

3. The system according to claim 2, characterized in that, in the event that the at least one user in the second user group is further affiliated to at least one associated user in the third user group, to at least one associated user in the fourth user group, and/or to at least one associated user in the fifth user group, the application cluster is further configured to distribute the collected fees among the at least one associated user in the second user group, the at least one associated user in the third user group, the at least one associated user in the fourth user group, and the at least one associated user in the fifth user group, according to a predetermined set of rules.

4. The system according to any of claims 1-3, characterized in that the application cluster is further configured to display an analytics dashboard in the second user interface using any information associated to the at least one user in the second user group, in the third user interface using any information associated to the at least one user in the third user group and the third set of affiliated users, in the fourth user interface using any information associated to the at least one user in the fourth user group and the second set of affiliated users, and in the fifth user interface using any information associated to the at least one user in the fifth user group and the first set of affiliated users.

5. The system according to any of claims 1-4, characterized in that the application cluster is configured to communicate with the first user group, the second user group, the third user group, the fourth user group, and the fifth user group via a web-based application.

6. The system according to claim 1, characterized in that the application cluster is configured to communicate with the first user group and the second user group via a mobile application.

7. The system according to claim 1 further comprising a queuing module, characterized in that said queuing module is configured to:
display a virtual waiting room via the first user interface, the second user interface, and/or the third user interface;
admit at least one user in the first user group to said virtual waiting room;
determine if the at least one user in the first user group has a paid appointment request;
add the at least one user in the first user group to a priority list, in the event that said at least one user has the paid appointment request;
provide a means for the at least one user in the first user group a means to settle payment, in the event that said at least one user has no paid appointment request and add said at least one user to a walk-in queue upon payment;
identify a first person in queue based on the priority list and the walk-in queue according to a predetermined set of rules; and
admit the first person in queue to a virtual consultation with the associated user in the second user group.

8. The system according to claim 7, characterized in that the application cluster is further configured to provide a real-time communication platform between the at least one user in the second user group and the at least one user in the first user group while in queue.

9. The system according to claim 8, characterized in that the application cluster is further configured to provide a real-time communication platform between the at least one user in the third user group and the at least one user in the first user group while in queue.

10. The system according to claim 1, characterized in that the first user group comprises a plurality of patients, the second user group comprises a plurality of healthcare practitioners, and the third user group comprises a plurality of clinic administrators, the fourth user group comprises a plurality of hospital managers and the fifth user group comprises a plurality of medical networks.

11. The system according to claim 1, characterized in that the first user interface is accessible via a first subdomain, the second user interface via a second subdomain, the third user interface via a third subdomain, the fourth user interface via a fourth subdomain, and the fifth user interface via a fifth subdomain.

12. The system according to claim 1, characterized in that the means for facilitating a real-time communication is in the form of a videoconference.

13. The system according to claim 10, further comprising a means for the at least one associated user in the second user group to manually mark an appointment as complete once a consultation took place instead of the videoconference.

14. The system according to claim 1, characterized in that the application cluster is further configured to provide a means for the at least one associated user in the second user group to transmit a plurality of medical documents to the at least one user in the first user group.

15. The system according to claim 14, characterized in that the plurality of medical documents comprises electronic prescriptions, laboratory tests requests, and medical notes.

16. The system according to claim 1, characterized in that the plurality of service orders issued to the first set of affiliated users comprises the issuance of a plurality of announcements or written memorandums and the assignment of a plurality of action items.

17. The system according to claim 16, characterized in that the plurality of action items are time-sensitive action items comprising doctor referrals, and removal or addition of affiliated users.

18. The system according to claim 1, characterized in that the plurality of service orders issued to the second set of affiliated users comprises actions for managing the second set of affiliated users and a plurality of service requests for referrals.

19. The system according to claim 1, further comprising a front desk system accessible to the users in the fourth user group via a fourth user interface and to users in the first user group via the first user interface.

20. The system according to claim 1, characterized in that the plurality of service orders issued to the third set of affiliated users comprises actions for managing the affiliated users, managing a plurality of appointment requests, and receiving and assigning of appointment referrals.

21. The system according to claim 1, characterized in that the application cluster is configured to grant API connection to at least one third-party application.

22. The system according to claim 1, characterized in that the application cluster is further configured to communicate with at least one user in a sixth user group via a sixth user interface.

23. The system according to claim 22, characterized in that at least one user in the sixth user group is linked to at least one user in the second user group, the sixth user group comprising a plurality of medical societies and the second user group comprising a plurality of healthcare practitioners.

24. The system according to claim 1, characterized in that the application cluster is further configured to communicate with at least one user in a seventh user group via a seventh user interface.

25. The system according to claim 24, characterized in that at least one user in the seventh user group is linked to at least one user in the fourth user group, the seventh user group comprising a plurality of medical partners and the fourth user group comprising a plurality of hospital managers.

26. The system according to claim 1, characterized in that the application cluster is further configured to communicate with at least one user in an eighth user group via an eighth user interface.

27. The system according to claim 26, characterized in that the application cluster is further configured monitor the activities of the plurality of information linked to the eighth user group, extract data from said activities according to a predetermined set of instructions, and visually present said data to the at least one user in the eighth user group.

28. The system according to claim 27, characterized in that said data are presented in the form of real-time reports, patient-and-doctor demographics, heat maps, revenue reports, and ratings information.

29. The system according to claim 1, characterized in that the application cluster is further configured to communicate with at least one user in a sixth user group via a sixth user interface and to provide a means for the at least one user the sixth user group to interact with at least one user in the second user group, characterized in that said at least one user in the sixth user group is a medical society administrator and said at least one user in the second user group is a healthcare practitioner.

30. The system according to claim 1, characterized in that the application cluster is further configured to communicate with at least one user in a seventh user group via a sixth user interface and to provide a means for the at least one user the seventh user group to interact with at least one user in the fourth user group, characterized in that said at least one user in the seventh user group is a medical partner and said at least one user in the fourth user group is a hospital manager.

31. The system according to claim 1, characterized in that the application cluster is further configured to communicate with at least one user in an eighth user group via an eighth user interface and to provide a means for the at least one user in the eighth user group to generate analytics from a plurality of information associated with said at least one user in the eighth user group.

32. A computer-implemented method for managing a plurality of healthcare services and networks comprising the steps of:
a. displaying a plurality of information to a first user group via a first user interface;
b. retrieving a plurality of appointment request information and a plurality of user information from at least one user in the first user group;
c. transmitting the plurality of appointment request information to an associated user in the second user group;
d. retrieving a response to the appointment request from the associated user in the second user group;
e. facilitating a real-time communication between the at least one user in the first user group and the associated user in the second user group;
f. identifying a first set of information, a second set of information, a third set of information, and a fourth set of information from the plurality of user information using a predetermined set of rules;
g. transmitting the first set of information to the associated user in the second user group;
h. determining if the associated user in the second user group is affiliated with at least one user in a third user group and/or with at least one user in a fourth user group and/or with at least one user in a fifth user group;
i. transmitting the second set of information to the at least one associated user in the third user group via a third user interface;
j. transmitting the third set of information to the at least one associated user in the fourth user group via a fourth user interface; and
k. transmitting the fourth set of information to the at least one associated user in the fifth user group via a fifth user interface.

33. The method according to claim 32 further comprising a step of retrieving a plurality of service orders from at least one user in the fifth user group via the fifth user interface and transmitting said service orders to a first set of affiliated users comprising a plurality of users from the fourth user group, a plurality of users from the third user group, and/or a plurality of users from the second user group.

34. The method according to any of claims 32 or 33 further comprising a step of retrieving a plurality of service orders from at least one user in the fourth user group via the fourth user interface and transmitting said service orders to a second set of affiliated users comprising a plurality of users from the third user group, and/or a plurality of users from the second user group.

35. The method according to any of claims 32-34 further comprising a step of retrieving a plurality of service orders from at least one user in the third user group via the third user interface and transmitting said service orders to a third set of affiliated users comprising a plurality of users from the second user group.

36. The method according to claim 32, further comprising a step of collecting fees from the at least one user in the first user group upon completion of the real-time communication and distributing the collected fees to the at least one associated user in the second user group.

37. The method according to claim 36, characterized in that, in the event that the at least one user in the second user group is further affiliated to at least one associated user in the third user group, to at least one associated user in the fourth user group, and/or to at least one associated user in the fifth user group, further comprises a step of distributing the collected fees among the at least one associated user in the second group, the at least one associated user in the third user group, the at least one associated user in the fourth user group and/or the at least one associated user in the fifth user group, according to a predetermined set of rules.

38. The method according to any of claims 32-35, further comprising a step of displaying an analytics dashboard in the second user interface using any information associated to the at least one user in the second user group, in the third user interface using any information associated to the at least one user in the third user group and the third set of affiliated users, in the fourth user interface using any information associated to the at least one user in the fourth user group and the second set of affiliated users, and in the fifth user interface using any information associated to the at least one user in the fifth user group and the first set of affiliated users.

39. The method according to claims 32 further comprising the steps of:
a. admitting at least one user in the first user group to a virtual waiting room, said virtual waiting room being accessible via the first user interface, the second user interface, and/or the third user interface;
b. determining if the at least one user in the first user group has a paid appointment request;
c. adding the at least one user in the first user group to a priority list, in the event that said at least one user has a paid appointment request;
d. providing a means for the at least one user in step (d) to settle payment for appointment request, in the event that said at least one user has no paid appointment request; and
e. adding the at least one user step in step (d) to a walk-in queue;
f. identifying a first person in queue based on the priority list and on the walk-in queue according to a predetermined set of rules; and
g. admitting the first person in queue to a virtual consultation with the associated user in the second user group.

40. The method according to claim 39, further comprising a step of providing a real-time communication platform between the at least one user in the second user group and the at least one user in the first user group while in queue.

41. The method according to claim 39 further comprising a step of providing a real-time communication platform between the at least one user in the third user group and the at least one user in the first user group while in queue.
